(19) United States
(12) Patent Application Publication
(10) Pub. No.: US 2024/0127499 A1
(43) Pub. Date: Apr. 18, 2024

(54) MAGNETIC RESONANCE IMAGE PROCESSING APPARATUS AND METHOD TO WHICH NOISE-TO-NOISE TECHNIQUE IS APPLIED

(71) Applicant: AIRS MEDICAL INC., Seoul (KR)

(72) Inventor: Jeewook KIM, Seoul (KR)

(21) Appl. No.: 17/790,840

(22) PCT Filed: May 3, 2022
(86) PCT No.: PCT/KR2022/006373

§ 371 Date: Jul. 5, 2022

(30) Foreign Application Priority Data
Aug. 12, 2021 (KR) 10-2021-0106677

(51) Int. Cl. G06T 11/00; G06T 5/70

(52) U.S. Cl. CPC G06T 11/006; G06T 5/70; G06T 2207/10088; G06T 2207/20081; G06T 2207/20084; G06T 2210/41; G06T 2211/441

(57) ABSTRACT

According to an embodiment of the present invention, there is provided a magnetic resonance image processing method to which a noise-to-noise technique is applied, the magnetic resonance image processing method including: acquiring an image obtained by scanning an object at least once; and, when images are acquired by scanning the object two or more times, training a first artificial neural network model by inputting any one of the images to the first artificial neural network model as an input and also inputting the other one of the images to the first artificial neural network model as a label.

### TECHNICAL FIELD

The present invention relates to a magnetic resonance image processing apparatus and method to which a noise-to-noise technique is applied, and more particularly to a magnetic resonance image processing apparatus and method for reducing magnetic resonance image acquisition time and improving the signal-to-noise ratio of a magnetic resonance image by applying a noise-to-noise technique to magnetic resonance image processing.

### BACKGROUND ART

In general, medical imaging machines are apparatuses that acquire information about the body of a patient and provide an image. Medical imaging machines include X-ray machines, ultrasound diagnostic scanners, computed tomography scanners, magnetic resonance imaging (MRI) machines, etc.

A magnetic resonance image is an image acquired by imaging the density and physical/chemical properties of atomic nuclei by generating nuclear magnetic resonance in the atomic nuclei of hydrogen in the human body using a magnetic field and non-ionizing radiation that are harmless to the human body. Magnetic resonance imaging machines occupy an important position in the field of diagnosis using medical images because the imaging conditions thereof are relatively free and they provide images including various types of diagnostic information from soft tissues and having desirable contrast.

Meanwhile, the imaging performed by such magnetic resonance imaging machines may take time ranging from about 20 minutes to about 1 hour depending on an imaging target region and the type of magnetic resonance image. In other words, a drawback arises in that the imaging time of the magnetic resonance imaging machines is longer than those of other types of medical imaging machines. This drawback may impose a burden regarding imaging on a patient. In particular, the drawback makes it difficult to apply magnetic resonance imaging to a patient with claustrophobia. Therefore, technologies for shortening imaging time have been developed until recently, and also, there is a demand for an improvement in image quality.

### SUMMARY

### Technical Problem

A magnetic resonance image processing apparatus and method to which a noise-to-noise technique is applied according to embodiments of the present invention output an output image by using an artificial neural network model trained by inputting any one of a plurality of images, acquired by scanning the same object, as an input and also inputting the other one as a label, thus intending to reduce image acquisition time and acquiring a magnetic resonance image having reduced noise.

In addition, a magnetic resonance image processing apparatus and method to which a noise-to-noise technique is applied according to embodiments of the present invention output an output image by using an artificial neural network model trained by inputting any one of a plurality of images, acquired by dividing fully sampled data, obtained by scanning the same object, into two pieces of sub-sampled data, as an input and also inputting the other one as a label, thus intending to reduce image acquisition time and acquiring a magnetic resonance image having reduced noise.

### Technical Solution

According to an embodiment of the present invention, there is provided a magnetic resonance image processing method to which a noise-to-noise technique is applied, the magnetic resonance image processing method including: acquiring an image obtained by scanning an object at least once; and, when images are acquired by scanning the object two or more times, training a first artificial neural network model by inputting any one of the images to the first artificial neural network model as an input and also inputting the other one of the images to the first artificial neural network model as a label.

In the present embodiment, there is provided the magnetic resonance image processing method, wherein when an output image is generated using the trained first artificial neural network model, an image having less noise than at least any one of an output image generated from a fully sampled magnetic resonance signal and an output image generated from a sub-sampled magnetic resonance signal by using a parallel imaging technique is output.

In another embodiment of the present invention, there is provided the magnetic resonance image processing method, including: when an image obtained by scanning the object once is acquired: acquiring a plurality of images by dividing the image into a first image for a magnetic resonance signal formed on even-numbered lines arranged based on a phase encoding direction and a second image for a magnetic resonance signal formed on odd-numbered lines arranged based on the phase encoding direction; and training a second artificial neural network model by inputting any one of the first and second images to the second artificial neural network model as an input and also inputting the other one of the first and second images to the second artificial neural network model as a label.

In the present embodiment, there is provided the magnetic resonance image processing method, wherein when an output image is generated using the trained second artificial neural network model, an image having less noise than at least any one of an output image generated from a fully sampled magnetic resonance signal and an output image generated from a sub-sampled magnetic resonance signal by using a parallel imaging technique is output.

### Advantageous Effects

The magnetic resonance image processing apparatus and method to which a noise-to-noise technique is applied according to embodiments of the present invention output an output image by using the artificial neural network model trained by inputting any one of a plurality of images, acquired by scanning the same object, as an input and also inputting the other one as a label, so that there are effects in that image acquisition time can be reduced and a magnetic resonance image having reduced noise can be acquired.

In addition, the magnetic resonance image processing apparatus and method to which a noise-to-noise technique is applied according to embodiments of the present invention output an output image by using the artificial neural network model trained by inputting any one of a plurality of images, acquired by dividing fully sampled data, obtained by scanning the same object, into two pieces of sub-sampled data, as an input and also inputting the other one as a label, so that there are effects in that image acquisition time can be reduced and a magnetic resonance image having reduced noise can be acquired.

### DESCRIPTION OF DRAWINGS

FIG. 1 is a block diagram illustrating the configuration of a magnetic resonance image processing apparatus to which a noise-to-noise technique is applied according to an embodiment of the present invention;

FIG. 2 is a flowchart showing the sequence of a magnetic resonance image processing method to which a noise-to-noise technique is applied that is performed in a magnetic resonance image processing apparatus to which a noise-to-noise technique is applied according to an embodiment of the present invention;

FIG. 3 is a diagram illustrating an artificial neural network model according to an embodiment of the present invention;

FIG. 4 is a schematic diagram illustrating the differences between the sub-sampling and full sampling of a magnetic resonance signal according to an embodiment of the present invention;

FIG. 5 shows views illustrating aliasing, Gibbs-ringing, and white-dot artifacts according to an embodiment of the present invention;

FIG. 6 shows views illustrating smoothing, texture, and hallucination artifacts according to an embodiment of the present invention;

FIG. 7 is a flowchart showing the sequence of a magnetic resonance image processing method to which a noise-to-noise technique is applied that is performed in a magnetic resonance image processing apparatus to which a noise-to-noise technique is applied according to another embodiment of the present invention;

FIG. 8 is a diagram showing an output image generated using a fully sampled magnetic resonance signal according to an embodiment of the present invention; and

FIG. 9 is a diagram showing an output image generated using a first artificial neural network model or a second artificial neural network model according to an embodiment of the present invention.

### DETAILED DESCRIPTION

Embodiments of the present invention will be described in detail below with reference to the accompanying drawings so that those of ordinary skill in the art to which the present invention pertains can easily practice the present invention. However, the present invention may be implemented in various different forms, and is not limited to the embodiments described herein. Furthermore, in the drawings, portions not related to the description are omitted for the clear description of the present invention, and like reference numerals are assigned to like portions throughout the specification.

Throughout the specification, when a part is described as being connected to another part, this includes not only a case in which they are “directly connected” to each other but also a case in which they are “electrically connected” to each other with another element interposed therebetween. In addition, when a part is described as “including” a component, this means that the part may further include another component, rather than excluding another component, unless otherwise described.

In the present specification, each of the ‘server’ and the ‘system’ refers to a computer configured to include one or more pieces of memory (not shown), one or more computer processors (not shown), and one or more programs (not shown). In this case, one or more programs (hereinafter ‘pre-processing programs’) are configured to be stored in memory and to be executed by one or more processors. The one or more pieces of memory, the one or more computer processors, and the one or more programs may be located in the physically same apparatus, and may be directly connected to each other or be connected to each other over a communication network.

In the present specification, the ‘image’ may refer to multi-dimensional data composed of discrete image elements (e.g., pixels in a two-dimensional image or voxels in a three-dimensional image). For example, an image may include medical images acquired by a medical imaging machine such as a magnetic resonance imaging (MRI) machine, a computed tomography (CT) scanner, an ultrasonic scanner, or an X-ray machine.

In the present specification, the ‘object’ is a target for imaging, and may include a human, an animal, or a part thereof. For example, an object may include a part (organ) of the body or a phantom. The phantom refers to a volume material having a density and an effective atomic number considerably close to those of an organism, and may include a spherical phantom having properties similar to those of the body.

A magnetic resonance image (MRI) system is a system that acquires images of tomographic areas of an object by representing the intensity of a magnetic resonance (MR) signal for a radio frequency (RF) signal generated from a magnetic field having a specific intensity in the form of contrasts between light and darkness.

The MRI system allows a main magnet to form a static magnetic field, and aligns the magnetic dipole moment direction of a specific atomic nucleus of an object, located in the static magnetic field, in the direction of the static magnetic field. A gradient magnetic field coil may generate a gradient magnetic field by applying a gradient signal to the static magnetic field, thereby inducing a different resonance frequency for each portion of the object. An RF coil may radiate a magnetic resonance signal in accordance with the resonance frequency of a portion where an image is to be acquired. Furthermore, as the gradient magnetic field is formed, the RF coil may receive magnetic resonance signals of different resonance frequencies radiated from various portions of the object. The MRI system acquires an image by applying an image reconstruction technique to the magnetic resonance signals received through this step. In addition, the MRI system may reconstruct a plurality of magnetic resonance signals into image data by performing serial or parallel signal processing on the plurality of magnetic resonance signals received by multi-channel RF coils.

A magnetic resonance image processing apparatus 100 to which a noise-to-noise technique is applied according to an embodiment of the present invention will be described below.

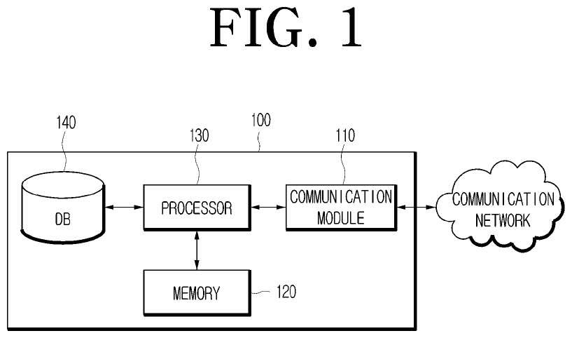

FIG. 1 is a block diagram illustrating the configuration of a magnetic resonance image processing apparatus to which a noise-to-noise technique is applied according to an embodiment of the present invention.

Referring to FIG. 1, the magnetic resonance image processing apparatus may include not only an MRI system capable of acquiring a magnetic resonance image by detecting a magnetic resonance signal on its own, but also an image processing apparatus for processing an image acquired from the outside, and a smartphone, a tablet personal computer (PC), a PC, a smart TV, a micro-server, a cloud server, other home appliances, and other mobile or non-mobile computing devices equipped with a function of processing a magnetic resonance image, but is not limited thereto. In addition, the magnetic resonance image processing apparatus may be a wearable device, such as a watch, glasses, a hairband or a ring, equipped with a communication function and a data processing function.

Furthermore, the magnetic resonance image processing apparatus 100 to which a noise-to-noise technique is applied according to an embodiment of the present invention may be directed to a magnetic resonance image processing apparatus that transmits and receives medical image data while communicating with a picture archiving and communication system (PACS) or a magnetic resonance imaging apparatus used in a medical institution and reconstructs magnetic resonance image data by using an artificial neural network model.

In addition, the magnetic resonance image processing apparatus 100 to which a noise-to-noise technique is applied according to an embodiment of the present invention may be implemented in the form of a cloud computing system. Cloud computing is a computing environment in which IT-related services such as data storage, networking, and the use of content can be used in an integrated manner through a server on the Internet. Alternatively, the magnetic resonance image processing apparatus 100 may be implemented as various types of computing systems capable of performing a magnetic resonance image processing method such as server computing, edge computing, and serverless computing.

More specifically, the magnetic resonance image processing apparatus 100 to which a noise-to-noise technique is applied according to an embodiment of the present invention may include a communication module 110, memory 120, a processor 130, and a database 140.

The communication module 110 provides a communication interface to the magnetic resonance image processing apparatus while operating in conjunction with a communication network. The magnetic resonance image processing apparatus 100 may transmit and receive data to and from a client terminal, a PACS terminal, and a PACS server to be described later by using the communication module 110. In this case, the communication module 110 may be a device including hardware and software necessary to transmit and receive signals, such as control signals or data signals, to and from another network device over a wired/wireless connection.

For example, the communication module 110 may perform communication via a local area network (LAN), Wideband Code Division Multiple Access (WCDMA), Long Term Evolution (LTE), wireless broadband (WiBro), the 5-th generation mobile communication (5G), ultra-wideband (UWB), ZigBee, radio frequency (RF) communication, a wireless LAN, Wireless Fidelity (Wi-Fi), near-field communication (NFC), Bluetooth, infrared communication, etc. However, this is an example, and various wired/wireless communication technologies applicable in the art may be used according to an embodiment to which the present invention is applied.

Meanwhile, in the present invention, the ‘terminal’ may be a wireless communication device with guaranteed portability and mobility, and may be one of all types of handheld-based wireless communication devices such as a smartphone, a tablet PC, and a notebook. Furthermore, the ‘terminal’ may be a wearable device, such as a watch, glasses, a hairband or a ring, equipped with a communication function and a data processing function. Moreover, the ‘terminal’ may be a wired communication device, such as a PC, that can be connected to another terminal or a server over a network.

A client terminal configured to control an medical imaging machine or manage the transmission of medical image data in conjunction with the medical imaging machine and a PACS terminal installed with a PACS program and configured to allow medical staff to view, process, and manage medical image data may be generally deployed in a medical institution. The client terminal may be a terminal on which a program providing a user interface (UI) configured to output user login, a worklist, and image processing details is installed. The PACS terminal may be a terminal on which a program providing a user interface configured to transmit medical image data and personal information data, stored in the PACS server, to the magnetic resonance image processing apparatus 100 and to receive the medical image data reconstructed via an artificial neural network model and then store it in the PACS server or output it to a display is installed.

The memory 120 may be a storage medium in which a program executed in the magnetic resonance image processing apparatus 100 is recorded. Furthermore, the memory 120 may perform a function of temporarily or permanently storing data that is processed by the processor 130. In this case, although the memory 120 may include volatile storage media or nonvolatile storage media, the scope of the present invention is not limited thereto.

The processor 130 may control the overall process of the program executed in the magnetic resonance image processing apparatus 100. In this case, the processor 130 may include all types of devices capable of processing data, such as a processor. In this case, for example, the ‘processor’ may refer to a data processing device embedded in hardware, which has circuits physically structured to perform functions represented by codes or instructions included in a program. Although as an example of the data processing device embedded in hardware as described above, processing devices such as a microprocessor, a central processing unit (CPU), a processor core, a multiprocessor, an application-specific integrated circuit (ASIC), and a field-programmable gate array (FPGA), may be enumerated, the scope of the present invention is not limited thereto.

The database 140 may be a component in which various types of data necessary for the magnetic resonance image processing apparatus 100 to execute the program are stored. For example, the database 140 may be a component in which a user list, a worklist, image processing information, protocol rules, medical image data, an artificial neural network model, and training data are stored.

A magnetic resonance image processing method to which a noise-to-noise technique is applied that is performed in a magnetic resonance image processing apparatus 100 to which a noise-to-noise technique is applied according to an embodiment of the present invention will be described below.

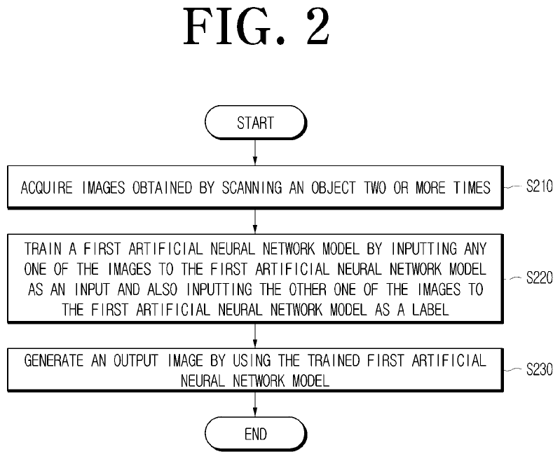

FIG. 2 is a flowchart showing the sequence of the magnetic resonance image processing method to which a noise-to-noise technique is applied that is performed in a magnetic resonance image processing apparatus to which a noise-to-noise technique is applied according to the embodiment of the present invention.

Referring to FIG. 2, there may be performed the step of acquiring an image obtained by scanning an object at least once. For example, step S210 of acquiring images obtained by scanning an object two or more times may be performed.

In magnetic resonance imaging, a plurality of scans may be performed to acquire an image having a high signal-to-noise ratio (SNR) for one object. Furthermore, the magnetic resonance image processing apparatus 100 may process images scanned a plurality of times so that a single image can be acquired. In this case, the processing of the images may be performed by a method of acquiring an image by summing the raw data of a plurality of images, imaging the summed raw data, and then removing noise from the imaged raw data, a method of acquiring an image by imaging the average data of the raw data of a plurality of images, or the like.

Data acquired from a magnetic resonance signal obtained from the magnetic resonance imaging apparatus may be referred to as raw data or k-space data. Imaging may mean acquiring an image from raw data or k-space data by using an inverse Fourier (IFFT) operation or an artificial neural network model.

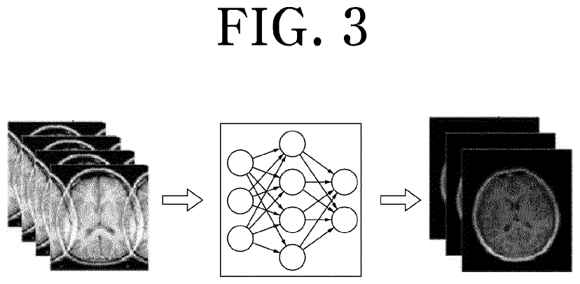

FIG. 3 is a diagram illustrating an artificial neural network model according to an embodiment of the present invention.

The artificial neural network model may be a set of algorithms for learning the correlation between at least one input magnetic resonance image and at least one label magnetic resonance image by using statistical machine learning results. The artificial neural network model may include at least one neural network. The neural network may include network models such as a Deep Neural Network (DNN), a Recurrent Neural Network (RNN), a Bidirectional Recurrent Deep Neural Network (BRDNN), a Multilayer Perceptron (MLP) network, and a Convolutional Neural Network (CNN), but is not limited thereto.

Thereafter, there may be performed step S220 of, when images obtained by scanning an object two or more times are acquired, training an artificial intelligence model by inputting any one of the images to a first artificial neural network model as an input and also inputting the other one to the first artificial neural network model as a label.

Thereafter, when an output image is generated using the trained first artificial intelligence model in step S230, there may be output an image having less noise than an output image generated from a fully sampled magnetic resonance signal and/or an output image generated from a sub-sampled magnetic resonance signal by using a parallel imaging technique.

For example, the first artificial neural network model is trained by inputting one of the images acquired by imaging the brain of an arbitrary object a plurality of times to the first artificial neural network model as an input image and also inputting the other one to the first artificial neural network model as a label image. Thereafter, there is generated an output image output by inputting an image, acquired by imaging the brain of another arbitrary object, to the first artificial neural network model as an input image. An output image acquired through the first artificial neural network model may have less noise than an output image acquired using a general magnetic resonance image processing apparatus.

The general magnetic resonance image processing apparatus may be a magnetic resonance image processing apparatus that generates an output image by imaging a fully sampled magnetic resonance signal through the performance of an inverse Fourier transform thereon. Alternatively, the general magnetic resonance image processing apparatus may be a magnetic resonance image processing apparatus that outputs a sub-sampled magnetic resonance signal, obtained through accelerated imaging, by improving the resolution of an output image using a parallel imaging technique.

In this case, the parallel imaging technique is a type of image reconstruction technique for acquiring high-accuracy k-space data and/or a high-accuracy magnetic resonance image, such as fully sampled k-space data and/or a magnetic resonance image, from the sub-sampled magnetic resonance signal and/or k-space data. In the performance of image reconstruction according to the parallel imaging technique, known technologies, i.e., Sensitivity Profiles From an Array of Coils for Encoding and Reconstruction in Parallel (SPACE RIP), Simultaneous acquisition of spatial harmonics (SMASH), Partially Parallel Imaging With Localized Sensitivities (PILS), Generalized Autocalibrating Partially Parallel Acquisitions (GRAPPA), and Iterative Self-consistent Parallel Imaging Reconstruction (SPIRiT), may be applied without limitations as long as they can be applied to the parallel imaging technique.

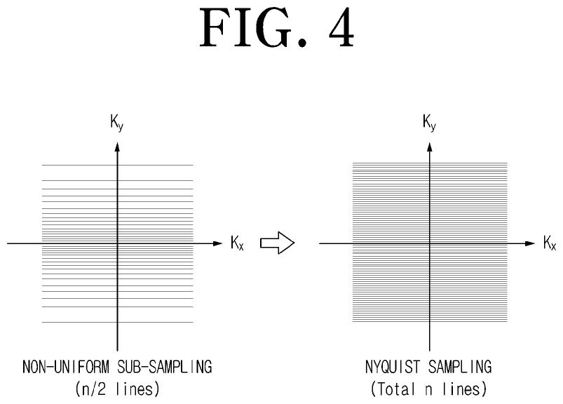

FIG. 4 is a schematic diagram illustrating the differences between sub-sampling and full sampling according to an embodiment of the present invention.

Referring to FIG. 4, a sub-sampled magnetic resonance signal may be a magnetic resonance signal sampled at a sampling rate lower than the Nyquist sampling rate. Furthermore, the sub-sampled magnetic resonance image is an image acquired by sampling a magnetic resonance signal at a sampling rate lower than the Nyquist sampling rate. Meanwhile, a fully sampled magnetic resonance image may be an image acquired by sampling k-space data at a sampling rate equal to or higher than the Nyquist sampling rate.

For example, the number of lines of a fully sampled magnetic resonance signal may be n, and the number of lines of a sub-sampled magnetic resonance signal may be n/2. In this case, when the degree of reduction in the number of sampling lines is a multiple of ½, the acceleration factor of magnetic resonance imaging may be considered to be 2. When the degree of reduction in the number of sampling lines is a multiple of ⅓ or a multiple of ¼, the acceleration factor may be considered to be 3 or 4.

Furthermore, sub-sampling methods may be divided into uniform sub-sampling and non-uniform sub-sampling. The uniform sub-sampling may be a method of performing sampling while maintaining the constant interval of lines to be sampled. In contrast, the non-uniform sub-sampling may refer to a method of performing more sampling while decreasing the interval of lines to be sampled in a direction toward the center of sampling target data and performing less sampling while increasing the interval of lines to be sampled in a direction away from the center.

Referring to FIG. 4, a phase encoding direction Ky may be a direction that extends parallel to a direction in which lines sampled in the process of sampling a magnetic resonance signal are stacked. In addition, a readout direction Kx may be a direction in which the sampled lines extend.

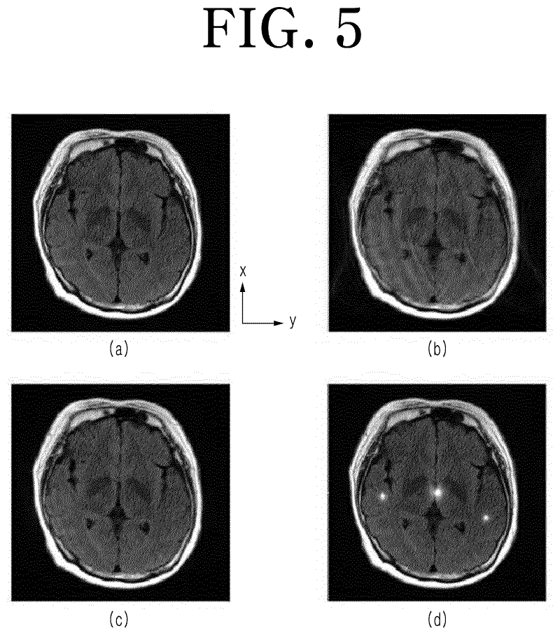

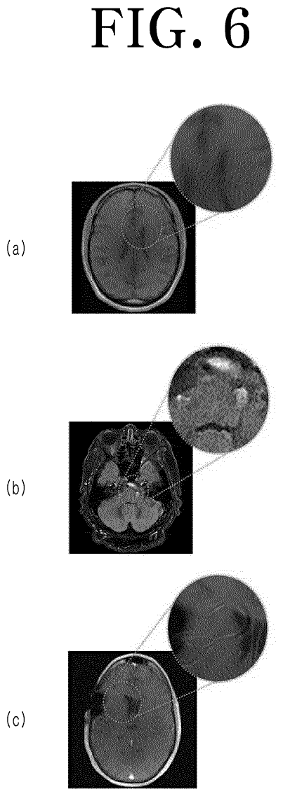

FIG. 5 shows views illustrating aliasing, Gibbs-ringing, and white-dot artifacts according to an embodiment of the present invention. FIG. 6 shows views illustrating smoothing, texture, and hallucination artifacts according to an embodiment of the present invention.

Referring to FIGS. 5 and 6, noise refers to an unnecessary signal generated for an electrical or mechanical reason, not a meaningful signal generated from an object to be scanned. An arbitrary noise value may be added to or subtracted from each pixel of an image. When a plurality of images is acquired by imaging the same object a plurality of times, pieces of noise between the plurality of images may be independent of each other. Such noise may include artifacts. There may be provided a magnetic resonance image processing method in which an artifact includes at least any one of an aliasing artifact, a Gibbs-ringing artifact, a white-dot artifact, a smoothing artifact, a texture artifact, and a hallucination artifact.

In this case, the aliasing artifact is an artifact (see FIG. 5(b)) in which a plurality of target images appears to overlap each other when a resulting image is compared with a target image (see FIG. 5(a)). Such an aliasing artifact may occur when data loss is severe in image reconstruction using an artificial neural network and/or when a field of view (FoV) is smaller than an imaged object. In this case, the target image is a magnetic resonance image reconstructed based on a fully sampled magnetic resonance signal.

The Gibbs-ringing artifact is an artifact in which a target image appears to fluctuate at the boundary thereof when a resulting image is compared with the target image (see FIG. 5(c)). Such a Gibbs-ringing artifact may occur when k-space data reconstructed using an artificial neural network may be subjected to data transformation and movement from a k-space domain to an image domain.

The white-dot artifact is an artifact (see FIG. 5(d)) in which a white dot appears on a portion of a target image when a resulting image is compared with the target image. Such a white-dot artifact may occur when an unnecessary bias component is generated in image reconstruction using an artificial neural network. In general, in an artificial neural network, a bias is generated or an activation function is included in neurons forming the artificial neural network in order to improve prediction performance. Due to the above-described structure of the artificial neural network, a bias may be formed in an output magnetic resonance image itself.

The smoothing artifact is an artifact in which the texture and details of the image appear to disappear (see FIG. 6(a)). Such a smoothing artifact may occur when the amount of information in input data is insufficient or the learning of training data is incomplete or fails with regard to an artificial neural network in the case where the artificial neural network is used for magnetic resonance image reconstruction and/or non-linear data optimization is used.

The texture artifact is an artifact in which the texture of an image appears to be altered (see FIG. 6(b)). For example, it can be seen from FIG. 6(b) that a zigzag-shaped texture that is not actually present is formed. The first reason why a texture artifact occurs is that when an image is reconstructed from data having a low signal-to-noise ratio (SNR) by using an artificial neural network, an image appears to be repeated horizontally due to aliasing and there are cases where this horizontal pattern does not disappear and remains in a reconstructed image. Second, in the case of a convolutional neural network (CNN), auxiliary maps are learned in each convolutional layer, in which case, when the learning is wrong, a wrong texture is used to reconstruct an image, resulting in a texture artifact.

The hallucination artifact is an artifact in which a structure that is not actually present appears present (see FIG. 6(c)). Referring to FIG. 6(c), a round structure at the center of an enlarged portion looks similar to a blood vessel shape on the lower right side of a brain image. The round structure is a structure that is not actually present, and is a shape generated by an artificial neural network. There may be various reasons for the occurrence of a hallucination artifact. The hallucination artifact may occur when an artificial neural network model determines that a sub-sampled k-space data input is data acquired from an object that is not actually present, and then reconstructs an image.

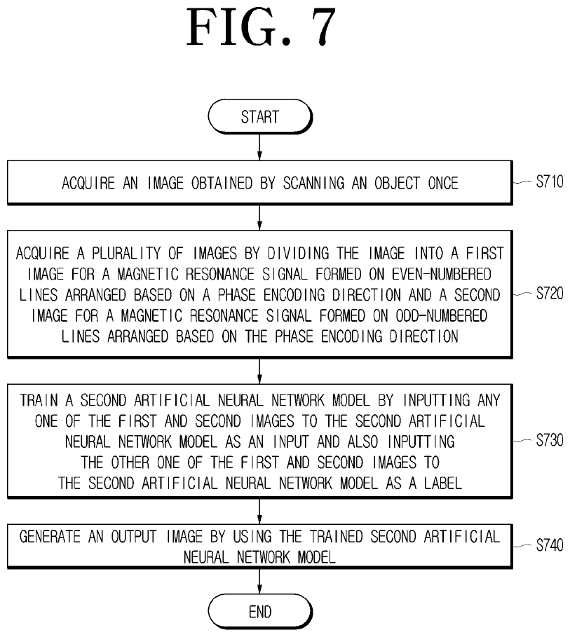

FIG. 7 is a flowchart showing the sequence of a magnetic resonance image processing method to which a noise-to-noise technique is applied that is performed in a magnetic resonance image processing apparatus to which a noise-to-noise technique is applied according to another embodiment of the present invention.

Referring to FIG. 7, in a magnetic resonance image processing method to which a noise-to-noise technique is applied according to another embodiment of the present invention, there may be performed step S710 of acquiring an image obtained by scanning an object once. Thereafter, there may be performed step S720 of acquiring a plurality of images by dividing the acquired image into a first image for portions formed on even-numbered sampled lines arranged based on a phase encoding direction Ky and a second image for portions formed on odd-numbered sampled lines arranged based on the phase encoding direction Ky.

Thereafter, there may be performed step S730 of training a second artificial neural network model by inputting any one of the first and second images to the second artificial neural network model as an input and also inputting the other one to the second artificial neural network model as a label.

In addition, when an output image is generated using the second learned artificial neural network model at step S740, an image having less noise than an output image generated from a fully sampled magnetic resonance signal and/or an output image generated from a sub-sampled magnetic resonance signal by using a parallel imaging technique may be output.

In addition, when the second artificial neural network model is used, an output image may be generated by using only one image as an input. The SNR of the output image may be similar to or improved from the SNR of the output image output by using a plurality of images as an input. In particular, the magnetic resonance image acquisition technique through the second artificial neural network model is a useful technique that can be applied to a case where there is only one scanned image of an object.

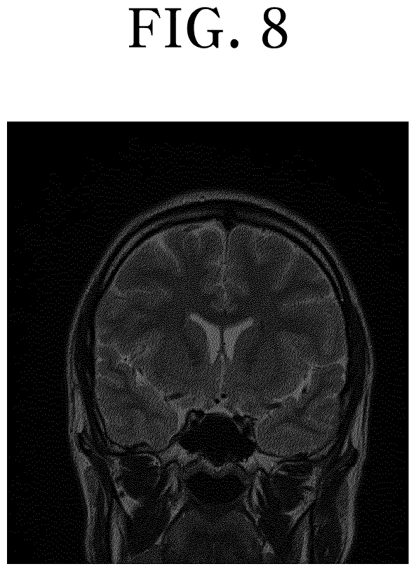

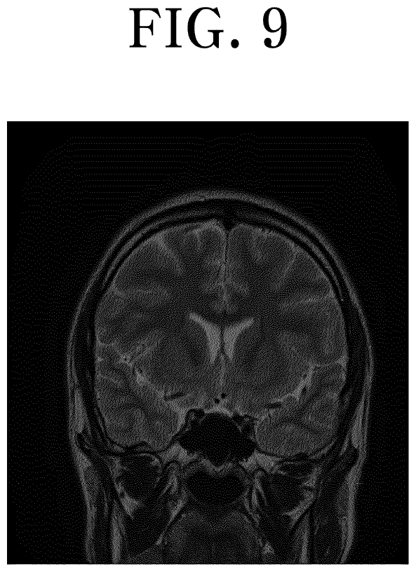

FIG. 8 is a diagram showing an output image generated using a fully sampled magnetic resonance signal according to an embodiment of the present invention. FIG. 9 is a diagram showing an output image generated using a first artificial neural network model or a second artificial neural network model according to an embodiment of the present invention.

Referring to FIGS. 8 and 9, it can be seen that an output image (see FIG. 9) generated using a first artificial neural network model according to an embodiment of the present invention or a second artificial neural network model according to another embodiment has less noise than an output image (see FIG. 8) generated using a fully sampled magnetic resonance signal.

The above-described magnetic resonance image processing apparatus 100 and method to which a noise-to-noise technique is applied according to embodiments of the present invention output an output image by using the artificial neural network model trained by inputting any one of a plurality of images, acquired by scanning the same object, as an input and also inputting the other one as a label, so that there are effects in that image acquisition time can be reduced and a magnetic resonance image having reduced noise can be acquired.

In addition, the magnetic resonance image processing apparatus 100 and method to which a noise-to-noise technique is applied according to embodiments of the present invention output an output image by using the artificial neural network model trained by inputting any one of a plurality of images, acquired by dividing fully sampled data, obtained by scanning the same object, into two pieces of sub-sampled data, as an input and also inputting the other one as a label, so that there are effects in that image acquisition time can be reduced and a magnetic resonance image having reduced noise can be acquired.

Meanwhile, the magnetic resonance image processing method to which a noise-to-noise technique is applied according to the embodiment of the present invention may be implemented in the form of a storage medium including instructions executable by a computer, such as a program module executed by a computer. Computer-readable media may be any available media that can be accessed by a computer, and include both volatile and nonvolatile media and removable and non-removable media. Furthermore, the computer-readable media may include computer storage media. The computer storage media include both volatile and nonvolatile media and removable and non-removable media implemented using any method or technology for the storage of information such as computer-readable instructions, data structures, program modules or other data. Although the method and system of the present invention have been described in connection with the specific embodiments, some or all of their components or operations may be implemented using a computer system having a general-purpose hardware architecture.

The above description is merely illustrative of the technical spirit of the present invention, and those of ordinary skill in the art to which the present invention pertains may make various modifications and alterations without departing from the essential features of the present invention. Accordingly, the embodiments disclosed in the present invention are not intended to limit the technical spirit of the present invention but are intended to describe the technical spirit of the present invention, and the scope of the technical spirit of the present invention is not limited by these embodiments. The scope of protection of the present invention should be interpreted by the following claims, and all technical spirits falling within the scope equivalent thereto should be interpreted as being encompassed in the scope of the present invention.

What is claimed is:

1. A magnetic resonance image processing method to which a noise-to-noise technique is applied, the magnetic resonance image processing method comprising:
acquiring an image obtained by scanning an object at least once; and
when images are acquired by scanning the object two or more times, training a first artificial neural network model by inputting any one of the images to the first artificial neural network model as an input and also inputting a remaining one of the images to the first artificial neural network model as a label.

2. The magnetic resonance image processing method of claim 1, wherein when an output image is generated using the trained first artificial neural network model, an image having less noise than at least any one of an output image generated from a fully sampled magnetic resonance signal and an output image generated from a sub-sampled magnetic resonance signal by using a parallel imaging technique is output.

3. The magnetic resonance image processing method of claim 1, comprising:
when an image obtained by scanning the object once is acquired:
acquiring a plurality of images by dividing the image into a first image for a magnetic resonance signal formed on even-numbered lines arranged based on a phase encoding direction and a second image for a magnetic resonance signal formed on odd-numbered lines arranged based on the phase encoding direction; and
training a second artificial neural network model by inputting any one of the first and second images to the second artificial neural network model as an input and also inputting a remaining one of the first and second images to the second artificial neural network model as a label.

4. The magnetic resonance image processing method of claim 3, wherein when an output image is generated using the trained second artificial neural network model, an image having less noise than at least any one of an output image generated from a fully sampled magnetic resonance signal and an output image generated from a sub-sampled magnetic resonance signal by using a parallel imaging technique is output.

5. A magnetic resonance image processing apparatus to which a noise-to-noise technique is applied, wherein the magnetic resonance image processing apparatus acquires an image obtained by scanning an object at least once, and, when images are acquired by scanning the object two or more times, trains a first artificial neural network model by inputting any one of the images to the first artificial neural network model as an input and also inputting a remaining one of the images to the first artificial neural network model as a label.
